SEQUENCE LISTING (22 sequences)

SEQ ID NO: 1 - protein, 58 aa, Artificial Sequence
  Type 2 Collagen binding protein
  Xaa (10)..(10): Xaa is selected from S or Q
  Xaa (14)..(14): Xaa is selected from S or Q
  Xaa (14)..(14): Xaa is selected from S or K
  Xaa (28)..(28): Xaa is selected from H or N
  Xaa (32)..(32): Xaa is selected from R or Q
  Xaa (35)..(35): Xaa is selected from S or R
  Xaa (42)..(42): Xaa is selected from T or P
  Xaa (43)..(43): Xaa is selected from H or T
  Xaa (44)..(44): Xaa is selected from I or V
  Xaa (46)..(46): Xaa is selected from G or T
  Xaa (47)..(47): Xaa is selected from Q or T
  Xaa (50)..(50): Xaa is selected from Q or H
  Xaa (54)..(54): Xaa is selected from S or D
IAAKFDEAQXAADXEILHLPNLTEQQRXYFRXWLXDDPSVSXXXLXXAQXLNDXQAPK

SEQ ID NO: 2 - protein, 58 aa, Artificial Sequence
  Type II collagen binding protein
IAAKFDEAQSAADSEILHLPNLTEQQRHYFRRWLSDDPSVSTHILTQAQHLNDDQAPK

SEQ ID NO: 3 - protein, 58 aa, Artificial Sequence
  Type II collagen binding protein
IAAKFDEAQQAADKEILHLPNLTEQQRNYFRQWLRDDPSVSPTVLGTAQQLNDSQAPK

SEQ ID NO: 4 - protein, 116 aa, Artificial Sequence
  Type 2 Collagen binding protein (dimer of SEQ ID NO 2)
IAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPK

SEQ ID NO: 5 - protein, 116 aa, Artificial Sequence
  Type II collagen binding protein (dimer of SEQ ID NO 3)
IAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPK

SEQ ID NO: 6 - protein, 562 aa, Artificial Sequence
  fusion protein 213182
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPK

SEQ ID NO: 7 - protein, 592 aa, Artificial Sequence
  fusion protein 213340
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQSAADSEILHLPNLTEQQRHYFRRWLSDDPSVSTHILTQAQHLNDDQAPKGGGGSGGGGSGGGGSGGGGSIAAKFDEAQSAADSEILHLPNLTEQQRHYFRRWLSDDPSVSTHILTQAQHLNDDQAPKSAWSHPQFEK

SEQ ID NO: 8 - protein, 678 aa, Artificial Sequence
  fusion protein 213184
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPK

SEQ ID NO: 9 - protein, 688 aa, Artificial Sequence
  fusion protein 213185
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKSAWSHPQFEK

SEQ ID NO: 10 - protein, 562 aa, Artificial Sequence
  fusion protein 212710
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPK

SEQ ID NO: 11 - protein, 572 aa, Artificial Sequence
  fusion protein 212708
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKSAWSHPQFEK

SEQ ID NO: 12 - protein, 692 aa, Artificial Sequence
  fusion protein 212709
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKGGGSIAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKSAWSHPQFEK

SEQ ID NO: 13 - protein, 562 aa, Artificial Sequence
  control fusion protein with n-terminal Aflibercept and c-terminal a dimer of a non-Type-II-Collagen binding scaffold
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQSAADSEILHLPNLTEEQRNAFRQSLSDDPSVSLEVLGEAQKLNDSQAPKIAAKFDEAQSAADSEILHLPNLTEEQRNAFRQSLSDDPSVSLEVLGEAQKLNDSQAPK

SEQ ID NO: 14 - protein, 58 aa, Artificial Sequence
  non Type II binding protein (PAdelFc)
IAAKFDEAQSAADSEILHLPNLTEEQRNAFRQSLSDDPSVSLEVLGEAQKLNDSQAPK

SEQ ID NO: 15 - protein, 430 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 16 - protein, 1486 aa, Artificial Sequence
  human Type II collagen
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 17 - protein, 4 aa, Artificial Sequence
  linker
GGGS

SEQ ID NO: 18 - protein, 5 aa, Artificial Sequence
  linker
GGGGS

SEQ ID NO: 19 - protein, 16 aa, Artificial Sequence
  linker
GGGGGSGGGGSGGGGS

SEQ ID NO: 20 - protein, 20 aa, Artificial Sequence
  linker
GGGGSGGGGSGGGGSGGGGS

SEQ ID NO: 21 - protein, 562 aa, Artificial Sequence
  fusion protein 217793
IAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 703 aa, Artificial Sequence
  fusion protein 218480
IAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSIAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKSAWSHPQFEK

(19) United States
(12) Patent Application Publication
(10) Pub. No.: US 2024/0209098 A1
(43) Pub. Date: Jun. 27, 2024

(54) FUSION PROTEINS WITH SPECIFITY FOR TYPE II COLLAGEN AND VEGF-A FOR THE TREATMENT OF EYE DISEASES

(71) Applicant: Navigo Proteins GmbH, Halle/Saale (DE)

(72) Inventors: Eva Bosse-Doenecke, Halle/Saale (DE); Manja Gloser-Braeunig, Halle/Saale (DE); Anja Katzschmann, Halle/Saale (DE); Ulrich Haupts, Halle/Saale (DE)

(73) Assignee: Navigo Proteins GmbH, Halle/Saale (DE)

(21) Appl. No.: 18/288,027

(22) PCT Filed: Sep. 11, 2021
(86) PCT No.: PCT/EP2021/081153

§ 371 Date: Oct. 23, 2023

(30) Foreign Application Priority Data
Apr. 23, 2021 (DE) 21170199.0

(51) Int. Cl. C07K 16/28; A61K 38/00; A61K 39/00; A61P 27/02

(52) U.S. Cl. CPC C07K 16/2863; A61P 27/02; A61K 38/00; A61K 2039/505; C07K 2319/35

(57) ABSTRACT

The present invention relates to new engineered fusion proteins with for use in treating disorders of the eye. In particular, the new fusion proteins are capable of binding both VEGF-A and Type II collagen. The fusion proteins comprise a subunit that specifically binds to Type II collagen which is a major component of the fibrillary structure of the vitreous humor of the eye. In addition to the Type II collagen binding protein, the new fusion proteins comprise a protein specific for VEGF-A and therapeutically effective in eye diseases. The invention further relates to the new fusion proteins for a use in the treatment of neovascular eye diseases.

### FIELD OF THE INVENTION

The present invention relates to new engineered fusion proteins with for use in treating disorders of the eye. In particular, the new fusion proteins are capable of binding both VEGF-A and Type II collagen. The fusion proteins comprise a subunit that specifically binds to Type II collagen which is a major component of the fibrillary structure of the vitreous humor of the eye. In addition to the Type II collagen binding protein, the new fusion proteins comprise a protein specific for VEGF-A and therapeutically effective in eye diseases. The invention further relates to the new fusion proteins for a use in the treatment of neovascular eye diseases.

### BACKGROUND OF THE INVENTION

Disorders of the eye are common diseases affecting many individuals. Neovascular eye diseases such as for example age-related macular degeneration (AMD) are the leading cause of irreversible vision loss among the aging population and affect more than 4 million individuals in the developed countries. AMD causes damage to the macula which is a spot centrally located on the retina of the eye and results in loss of sharp, central vision. Other retinal neovascular diseases include diabetic retinopathy, retinal vein occlusion, and diabetic macular edema (DME). In diabetic retinopathy, a common complication in diabetes, damaged blood vessels of the light-sensitive tissue at the back of usually both eyes are the reason for impaired vision or even vision loss. After the development of diabetic retinopathy patients may be affected by diabetic macular edema where fluids accumulate in the macula of the retina due to leaking blood vessels. Retinal vein occlusion is an eye disease where veins in the retina are blocked resulting in blurry vision or sudden blindness.

Current procedures for the treatment of eye diseases such as AMD include proteins like Ranibizumab (tradename Lucentis®), Bevacizumab (tradename Avastin®), or Aflibercept (tradename Eylea©). Although a topical application of these drugs is possible, a disadvantage of the topical application is a very fast wash out from the eye and accordingly, a very low fraction of drug uptake. An alternative to topical applications to the eye are injections; however, the treatment of eye diseases requires frequent intraocular injections of drugs as often as every 1-2 months, into one or both eyes, often for life. This sums up to 6-12 injections per year. Pharmaceutical interventions through injections into the vitreous are inconvenient for patients since they are risky and painful for the patients.

Thus, needless to say that existing therapies for eye diseases have significant disadvantages for patients. They are burdensome for patients in terms of pain, cost, time, and risk. There is a strong need to relief patients from the imperative frequent and burdening treatments of eye diseases.

Due to significant limitations of current therapies for eye diseases, there was a need to provide novel proteins for the treatment of ocular diseases with improved properties, in particular in view of prolonged availability in the eye to reduce the painful frequent injection. Accordingly, there is a need in this field to obtain novel proteins suitable for more effective approaches for the therapy of eye diseases.

One objective of the present invention is the provision of molecules for anchoring a therapeutic protein effective for eye diseases near the diseased ocular tissue to prolong the beneficial impact of the therapeutic protein for eye diseases.

The present invention provides artificial fusion proteins of VEGF-A binding proteins and collagen-2 binding proteins that are particularly well-suited for the treatment of eye diseases but overcome the disadvantages of current approaches.

The above-described objectives and advantages are achieved by the subject-matters of the enclosed claims. The present invention meets the needs presented above by providing examples for fusion proteins. Preferred embodiments of the invention are included in the claims as well as in the following description, examples and figures. The above overview does not necessarily describe all problems solved by the present invention.

### SUMMARY OF THE INVENTION

The present disclosure provides the following items 1 to 15, without being specifically limited thereto:
1. A fusion polypeptide capable of binding to VEGF-A and Type II collagen that comprises at least two subunits, wherein
   a. the first subunit is a binding protein specific for VEGF-A, and
   b. the second subunit is a binding protein specific for Type II collagen, preferably wherein the second subunit comprises at least one amino acid sequence with at least 95% sequence identity to SEQ ID NO: 1.
2. The fusion polypeptide of item 1, wherein the first subunit comprises a full-length immunoglobulin or an antigen-binding domain thereof or an extracellular domain of a receptor or fragments thereof having binding affinity for VEGF-A.
3. The fusion polypeptide of items 1 to 2, wherein the first subunit is selected from Aflibercept, Ranibizumab, Bevacizumab, or Brolucizumab, or fragments thereof, or biosimilars thereof.
4. The fusion polypeptide of any one of items 1-3, wherein the second subunit comprises at least one amino acid sequence with at least 95% sequence identity to SEQ ID NO: 1.
5. The fusion polypeptide of any one of items 1-4, wherein the second subunit comprises at least one amino acid sequence with at least 95% sequence identity to SEQ ID NO: 2 or with at least 95% sequence identity to SEQ ID NO: 3.
6. The fusion polypeptide of any one of items 1-5, wherein the second subunit comprises a multimer of an amino acid sequence with at least 95% sequence identity to SEQ ID NO: 1.
7. The fusion polypeptide of any one of items 1-6, wherein the fusion polypeptide does not bind to Type I collagen, Type III collagen, and/or Type V collagen.
8. The fusion polypeptide of any one of items 1-7, wherein the first subunit can be linked to the second subunit via a linker.
9. The fusion polypeptide of any one of items 1-8, wherein the fusion polypeptide comprises at least two subunits in N- to C-terminal orientation, wherein
   a. the subunit at the N-terminus is specific for VEGF-A, and
   b. the subunit at the C-terminus is specific for Type II collagen.
10. The fusion polypeptide of any one of items 1-9, wherein the fusion polypeptide comprises at least two subunits in N- to C-terminal orientation, wherein
   a. the subunit at the N-terminus is specific for Type II collagen, and
   b. the subunit at the C-terminus is specific for VEGF-A.
11. The fusion polypeptide of any one of items 1-10 wherein the fusion polypeptide is specifically binding to human VEGF-A and human Type II collagen.
12. The fusion polypeptide according to any one of items 1-11 for use in the treatment of neovascular eye diseases
13. The fusion protein or polypeptide of any one of items 1-12 wherein the Type II collagen binding protein increases the half-life of the therapeutic protein at least 1.5 fold.
14. A pharmaceutical composition for the treatment of eye diseases comprising the fusion polypeptide as defined in any one of items 1-13 and a therapeutically acceptable carrier and/or diluent.
15. A method for the production of the fusion polypeptide as defined in any one of items 1-14 comprising culturing of a host cell under suitable conditions in order to obtain said fusion protein and optionally isolating said fusion polypeptide.

This summary of the invention does not necessarily describe all features of the present invention. Other embodiments become apparent from a review of the ensuing detailed description.

### BRIEF DESCRIPTION OF THE FIGURES

FIG. 1. Schematic drawing of a bispecific fusion protein of a Type II collagen binding protein with an VEGF-A binding protein. Black circles represent the Type II collagen binding protein(s). In grey: VEGF-A binding protein Aflibercept.

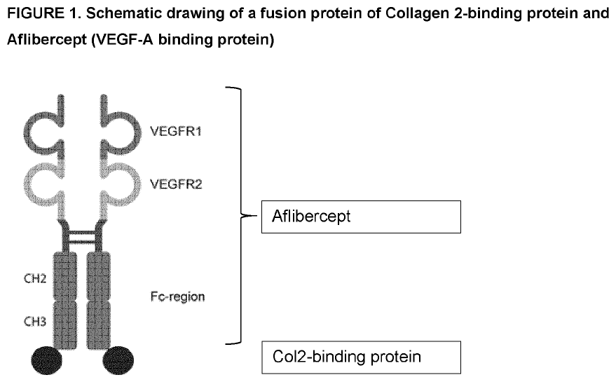

FIG. 2 shows the KD-determination of Type II collagen (Col II) binding proteins by ELISA with collagen Type II proteins of different species (human (h), rabbit (rb), pig (p)) and various collagen Types, like Type I collagen (Col I), Type III collagen (Col III), Type V collagen (Col V). Fusion proteins comprising Aflibercept and SEQ ID NO: 2 or SEQ ID NO: 3 (FIG. 2A: CID 213182/SEQ ID NO: 6, FIG. 2B: CID 213184/SEQ ID NO: 8, FIG. 2C: CID 212709/SEQ ID NO: 12) show a strong specific binding to Type II collagen with a KD of 2-3-digit pM.

FIG. 3. Immunohistochemistry staining of fusion protein 213184 on pig eye sections. 500 nM CID 213184 shows staining of inner limiting membrane (star) and vitreous body (arrow) whereas the control (TBS) does not show any staining.

FIG. 4. Average Retinal Leakage Score (Day 3 post challenge). Fusion proteins 213182 and 212710 (SEQ ID NOs: 6 and 10, respectively) and Aflibercept prevented the retinal vascular leakage induced by VEGF administration compared to vehicle pretreatment.

### DETAILED DESCRIPTION OF THE INVENTION

collagens are structural proteins forming three-dimensional meshworks. Type II collagen forms homotrimers which form higher order oligomers resulting in fibrillary structures. The fusion proteins of the invention are specifically bound to the major component of the fibrillary structure of the vitreous humor of the eye, Type II collagen. The other part of the novel fusion protein or fusion polypeptide of the invention is a VEGF-A binding protein, in particular a VEGF-A binding protein that is a therapeutic protein effective in the treatment of eye diseases. Due to the anchoring of the fusion protein in the eye by binding to Type II collagen, the local residence time of the therapeutic protein is enhanced so that less frequent painful treatments of eye diseases are required. The novel fusion proteins of the invention enable fewer medical interventions and safer therapies in eye diseases and improve quality of life for patients.

The present inventors have developed a solution to meet the ongoing need in the art by providing fusion proteins comprising Type II collagen specific proteins and VEGF-A specific proteins. The Type II collagen specific proteins are based on a small artificial triple helix protein and are functionally characterized by high affinity for Type II collagen. The fusion proteins of the invention provide molecular formats with favorable physicochemical properties, in particular, they are stable and may broaden therapeutic options. Therapeutic proteins for eye diseases, if fused to the Type II collagen binding protein as disclosed herein, may have a longer duration of action in eye diseases since the clearance of the therapeutic protein from the vitreous body is decreased and thereby the half-life is extended. The fusion proteins provided herein, the compositions and methods allow for retention of therapeutic proteins for eye diseases for a longer period of time. Further, this enhances patient acceptance and quality of life and is an improvement over current treatment strategies.

Before the present invention is described in more detail below, it is to be understood that this invention is not limited to the particular methodology, protocols and reagents described herein as these may vary. It is also to be understood that the terminology used herein is for the purpose of describing particular aspects and embodiments only and is not intended to limit the scope of the present invention, which is reflected by the appended claims. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new fusion molecules for use in therapy of various eye diseases. Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims, which follow, unless the context requires otherwise, the word “comprise”, and variants such as “comprises” and “comprising”, was understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, GenBank Accession Number sequence submissions etc.) may be cited throughout the present specification. Nothing herein is to be construed as an admission that the invention is not entitled to antedate such disclosure by virtue of prior invention. All sequences referred to herein are disclosed in the attached sequence listing that, with its whole content and disclosure, forms part of the disclosure content of the present specification.

### General Definitions of Important Terms Used in the Application

The term “fusion protein” relates to a protein comprising at least a first protein joined genetically to at least a second protein. A fusion protein is created through joining of two or more genes that originally coded for separate proteins. Fusion proteins may further comprise additional domains that are not involved in binding of the target, such as but not limited to, for example, multimerization moieties, polypeptide tags, polypeptide linkers, half-life extending moieties.

The terms “protein” and “polypeptide” refer to any chain of two or more amino acids linked by peptide bonds and does not refer to a specific length of the product. Thus, “peptides”, “protein”, “amino acid chain”, or any other term used to refer to a chain of two or more amino acids, are included within the definition of “protein”, and the term “protein” may be used instead of, or interchangeably with, any of these terms. The term “protein” is also intended to refer to the products of post-translational modifications of the polypeptide which are well known in the art.

The term “VEGF” or “vascular endothelial growth factor” is a human vascular endothelial growth factor. VEGF-A (uniprot Accession Number P15692) exists as a number of different isotypes which are generated both by alternative splicing and proteolysis, for example, VEGF-206, VEGF-189, VEGF-165, and VEGF-121. The isoforms are all biologically active as dimers. VEGF-A herein means any of the natural isoforms or natural variants or induced variants having at least a sequence identity of at least 80%, 85%, 90%, 95%, 96% or 97% or more, or 100% to a natural isoform or natural variant. In some embodiments, VEGF-A is human VEGF-A. In some embodiments, VEGF-A is human VEGF-A.

The term “Type II collagen” or “collagen 2” or “Type II collagen alpha I chain” as used herein refers to uniprot Accession Number P02458.2, amino acids 182 to 1241 (SEQ ID NO: 16). The term “Type II collagen” comprises all polypeptides which show a sequence identity of at least 80%, 85%, 90%, 95%, 96% or 97% or more, or 100% to SEQ ID NO: 16 and have the functionality of Type II collagen. In particular, the term comprises Type II collagen from other mammalian species with amino acid sequence identity of at least 80%, 85%, 90%, 95%, 96% or 97% or more, or 100% to SEQ ID NO: 16. In accordance with the reference to type II collagen identified under uniprot Accession Number P02458.2, in preferred embodiments the term “type II collagen” as used herein means human type II collagen.

The terms “Type II collagen binding protein” or “protein with binding specificity for Type II collagen” or “Type II collagen specific binding protein” or “Col2 BP” refers to a protein with high affinity binding to Type II collagen.

The terms “protein with binding specificity for VEGF-A” or “VEGF-A binding protein” or “VEGF-A specific binding protein” refer to a protein with high affinity binding to VEGF-A.

The term “modification” or “amino acid modification” refers to a substitution, a deletion, or an insertion of an amino acid at a particular position in a parent polypeptide sequence by another amino acid. Given the known genetic code and recombinant and synthetic DNA techniques the skilled scientist can readily construct DNAs encoding the amino acid variants.

The term “substitution” is understood as exchange of an amino acid by another amino acid. The term “insertion” comprises the addition of amino acids to the original amino acid sequence.

The terms “binding affinity” and “binding activity” may be used herein interchangeably, and they refer to the ability of a polypeptide of the invention to bind to another protein, peptide, or fragment or domain thereof. Binding affinity is typically measured and reported by the equilibrium dissociation constant (K_{D}), which is used to evaluate and rank order strengths of bimolecular interactions.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide. Methods for sequence alignment and sequence comparison algorithms are well known in the art. For example, for determining the extent of an amino acid sequence identity of an arbitrary polypeptide relative to the reference amino acid sequence, the SIM Local similarity program is preferably employed. For multiple alignment analysis, ClustalW as known to someone skilled in the art is preferably used.

The terms “therapeutic protein for eye diseases” or “protein therapeutically effective in eye diseases” may be interchangeably and relate to a protein that is used for therapies of eye diseases. Thus, a therapeutic protein for eye diseases is understood as a protein for use in treating a disorder that affects the eye. Therapeutic proteins for eye diseases might be classified based on pharmacological action, eg. group I: protein therapeutics with enzymatic or regulatory activity (replacement of a protein that is deficient or abnormal; augmentation or inhibition of an existing pathway; provides a novel function or activity); group II: protein therapeutics with special targeting activity (interferes with a molecule or signaling pathway, delivers other compounds or proteins such as radionuclide, cytotoxic drug, or effector protein). Another classification of therapeutic proteins is based on molecular types: antibody based binders, non-immunoglobulin scaffold based binders, enzymes, growth factors, hormones, interferons, and interleukins as well as fusions of these such as Fc-fusions or fusions with half-life extending moieties. Yet a further classification is based on molecular mechanism: binding non-covalently to target (antibodies and non-immunoglobulin scaffolds and others), pathway activation or pathway inhibition, or enzymatic or transport or structural.

The term “half-life” refers to the time that is needed for the concentration of a therapeutic protein for eye diseases to be reduced by one-half.

The term “local residence time” is the time that the therapeutic protein for eye diseases resides in the eye.

### DETAILED DESCRIPTION OF THE EMBODIMENTS OF THIS INVENTION

The fusion proteins of the invention comprise, essentially consist of or consist of at least two subunits wherein the first subunit is specific for VEGF-A and the second subunit is specific for Type II collagen.

### Structural Characterization of the Type II Collagen Binding Subunit of the Fusion Protein.

In various embodiments, a Type II collagen binding protein has at least 95% amino acid sequence identity to

IAAKFDEAQX₁₀AADX₁₄EILHLPNLTEQQRX₂₈YFRX₃₂WLX₃₅DDPSVSX₄₂X₄₃X₄₄LX₄₆X₄₇AQX₅₀LN DX₅₄QAPK (SEQ ID NO: 1) where, individually of each other, X₁₀ is selected from S or Q, X₁₄ is selected from S or K, X₂₈ is selected from H or N, X₃₂ is selected from R or Q, X₃₅ is selected from S or R, X₄₂ is selected from T or P, X₄₃ is selected from H or T, X₄₄ is selected from I or V, X₄₆ is selected from T or G, X₄₇ is selected from Q or T, X₅₀ is selected from H or Q, and X₅₄ is selected from S or D. In some embodiments, a Type II collagen binding protein has at least 95% amino acid sequence identity to SEQ ID NO: 1: wherein, individually of each other, position 25 is Q, position 29 is Y, and position 33 is W.

In various embodiments, a Type II collagen binding protein has at least 95% amino acid sequence identity to

IAAKFDEAQX₁₀AADX₁₄EILHLPNLTEQQRX₂₈YFRX₃₂WLX₃₅DDPSVSX₄₂X₄₃X₄₄LX₄₅X₄₇AQX₅₀LN DX₅₄QAPK (SEQ ID NO: 1) wherein X₁₀ is selected from S or Q, X₁₄ is selected from S or K, X₂₈ is selected from H or N, X₃₂ is selected from R or Q, X₃₅ is selected from S or R, X₄₂ is selected from T or P, X₄₃ is selected from H or T, X₄₄ is selected from I or V, X₄₆ is selected from T or G, X₄₇ is selected from Q or T, X₅₀ is selected from H or Q, and X₅₄ is selected from S or D. In some embodiments, a Type II collagen binding protein has at least 95% amino acid sequence identity to SEQ ID NO: 1 wherein position 25 is Q, position 29 is Y, and position 33 is W.

The amino acid identity to SEQ ID NO: 1 to SEQ ID NO: 12, SEQ ID NO: 14 is at least 75%, at least 80%, at least 85%, at least 90%, but not more than 92%. SEQ ID NO: 14 does not bind to Type II collagen. At least substitutions E25Q, A29Y, and S33W in SEQ ID NO: 14 are required for Type II collagen binding. Further substitutions are selected from one or more of S10Q, S14K, N28H, Q32R, S35R, L42P, L42T, E43H, E43T, V44I, G46T, E47Q, E47T, K50H, K50Q, S54D, preferably additional substitutions are selected from 5, 6, 7, 8, or 9 of S10Q, S14K, N28H, Q32R, S35R, L42P, L42T, E43H, E43T, V44I, G46T, E47Q, E47T, K50H, K50Q, S54D.

In various embodiments, a Type II collagen binding protein has at least 95% amino acid sequence identity to IAAKFDEAQSAADSEILHLPNLTEQQRHYFRRWLSDDPSVSTHILTQAQHLNDDQAPK (SEQ ID NO: 2).

The Type II collagen binding protein of the present invention as disclosed herein has at least 95%, 96%, 97%, 98%, or 99% amino acid sequence identity to the amino acid sequence of SEQ ID NO: 2. In some embodiments, the Type II collagen binding protein of the present invention as disclosed herein has at least 95%, 96%, 97%, 98%, or 99% amino acid sequence identity to the amino acid sequence of SEQ ID NO: 2 wherein position 25 is Q, position 29 is Y, and position 33 is W (underlined in above shown sequence).

The amino acid identity to SEQ ID NO: 2 to SEQ ID NO: 14 is 79%. SEQ ID NO: 14 does not bind to Type II collagen. At least substitutions E25Q, A29Y, and S33W in SEQ ID NO: 14 are required for Type II collagen binding. Further substitutions are selected from one or more of N28H, Q32R, L42T, E43H, V44I, G46T, E47Q, K50H, S54D.

In various embodiments, the Type II collagen binding protein has at least 95% amino acid sequence identity to SEQ ID NO: 3. The Type II collagen binding protein of the present invention as disclosed herein has at least 95%, 96%, 97%, 98%, or 99% amino acid sequence identity to the amino acid sequence of IAAKFDEAQQAADKEILHLPNLTEQQRNYFRQWLRDDPSVSPTVLGTAQQLNDSQAPK (SEQ ID NO: 3). In some embodiments, the Type II collagen binding protein of the present invention as disclosed herein has at least 95%, 96%, 97%, 98%, or 99% amino acid sequence identity to the amino acid sequence of SEQ ID NO: 3, provided that position 25 is Q, position 29 is Y, and position 33 is W (underlined in above shown sequence).

The amino acid identity to SEQ ID NO: 3 to SEQ ID NO: 14 is 86%. SEQ ID NO: 14 does not bind to Type II collagen. At least substitutions E25Q, A29Y, and S33W in SEQ ID NO: 14 are required for Type II collagen binding. Further substitutions are selected from one or more of S10Q, S14K, S35R, L42P, E43T, E47T, K50Q.

In accordance with the above, in various embodiments of the present invention, a type II collagen binding protein has at least 95% amino acid sequence identity to any of SEQ ID NOs: 1, 2 or 3, wherein the type II collagen protein has (i) a glutamine (Q) at the position corresponding to position 25 in any of SEQ ID NOs: 1, 2, and 3; (ii) a tyrosine (Y) at the position corresponding to position 29 in any of SEQ ID NOs: 1, 2, and 3; and (iii) a tryptophan (W) at the position corresponding to position 33 in any of SEQ ID NOs: 1, 2, and 3.

In various other embodiments of the present invention, a type II collagen binding protein has at least 95% amino acid sequence identity to any of SEQ ID NOs: 1, 2 or 3, wherein the type II collagen protein has (i) a glutamine (Q) at the position corresponding to position 25 in any of SEQ ID NOs: 1, 2, and 3; (ii) a tyrosine (Y) at the position corresponding to position 29 in any of SEQ ID NOs: 1, 2, and 3; and (iii) a tryptophan (W) at the position corresponding to position 33 in any of SEQ ID NOs: 1, 2, and 3, and wherein the type 11 collagen protein has a binding affinity (KD) of less than 1 μM for type II collagen, as described elsewhere herein.

In various embodiments of the present invention, a type II collagen binding protein as disclosed herein may have a deletion of one or more, preferably two, amino acid residues at the N-terminus of any of SEQ ID NOs: 1 to 5 and 14. In preferred embodiments of dimeric and multimeric type II collagen binding proteins disclosed herein, only the monomer located at the N-terminus of the dimer or multimer may have a deletion of one or more, preferably two, amino acid residues at the N-terminus of the monomer.

In various embodiments of the present invention, a (monomer) type II collagen binding protein as disclosed herein comprises at least 47 amino acid residues of any of SEQ ID NOs: 1 to 5 and 14.

In various other embodiments of the present invention, a monomer type II collagen binding protein as disclosed herein comprises at least 48 or 49 amino acid residues of any of SEQ ID NOs: 1 to 5 and 14. In still other embodiments of the present invention, a monomer type II collagen binding protein as disclosed herein comprises at least 50 or 51 amino acid residues of any of SEQ ID NOs: 1 to 5 and 14. In various preferred embodiments of the present invention, a monomer type II collagen binding protein as disclosed herein comprises at least 52 amino acid residues of any of SEQ ID NOs: 1 to 5 and 14. In various other preferred embodiments of the present invention, a monomer type II collagen binding protein as disclosed herein comprises at least 53 amino acid residues of any of SEQ ID NOs: 1 to 5 and 14. More preferably, a monomer type II collagen binding protein as disclosed herein comprises at least 54 or 55 amino acid residues of any of SEQ ID NOs: 1 to 5 and 14. In particularly preferred embodiments of the present invention, a monomer type 11 collagen binding protein as disclosed herein comprises at least 56 amino acid residues of any of SEQ ID NOs: 1 to 5 and 14. In various preferred embodiments of the present invention, a monomer type II collagen binding protein as disclosed herein comprises at least 57 or even 58 amino acid residues of any of SEQ ID NOs: 1 to 5 and 14.

In the fusion polypeptides of the present invention, the type II collagen binding protein may be located at the N-terminus or at the C-terminus of the fusion polypeptide. In various embodiments, the type II collagen binding protein is located at the N-terminus or at the C-terminus of the fusion polypeptide in head-to-tail orientation as described elsewhere herein. In various embodiments, the VEGF-A specific protein has a type II collagen binding protein as described herein fused to each the N-terminus and the C-terminus. The type II collagen binding proteins fused to the N-terminus and the C-terminus of the VEGF-A specific protein may or may not be identical. In various embodiments, the VEGF-A specific protein may have a dimer or a multimer of the type II collagen binding protein(s) as described elsewhere herein fused to its N-terminus and/or the C-terminus.

In various embodiments, a fusion polypeptide of the present invention comprises a binding protein for type II collagen comprising an amino acid sequence with at least 95% sequence identity to any one of the dimers of SEQ ID NOs: 4 and 5. In preferred embodiments, at least one of the two monomers of a type II collagen binding protein comprising an amino acid sequence with at least 95% sequence identity to any one of the dimers of SEQ ID NOs: 4 and 5, is a type II collagen binding protein having (i) a glutamine (Q) at the position corresponding to position 25 in any of SEQ ID NOs: 4 or 5; (ii) a tyrosine (Y) at the position corresponding to position 29 in any of SEQ ID NOs: 4 or 5; and (iii) a tryptophan (W) at the position corresponding to position 33 in any of SEQ ID NOs: 4 or 5. In various preferred embodiments, the at least one of the two monomers of a type II collagen binding protein has a binding affinity (K_{D}) of less than 1 μM for type II collagen, as described elsewhere herein.

Characterization of the VEGF-A specific subunit of the fusion protein. The fusion protein or fusion polypeptide as disclosed herein includes a VEGF-A specific binding protein. Accordingly, as described herein, a fusion polypeptide of the invention comprises a protein with binding specificity for VEGF-A. As described herein, a protein with binding specificity for VEGF-A is capable of inhibiting the activity of VEGF-A. In particular, as disclosed herein, a protein with binding specificity for VEGF-A prevents binding of VEGF-A to the receptors VEGFR-1 (also known as Flt-1) and/or VEGFR-2 (also known as KDR or Flk-1).

In various embodiments the VEGF-A specific protein may be selected from a full-length immunoglobulin or an antigen-binding domain thereof or an extracellular domain of a receptor or fragments thereof having binding affinity for VEGF-A. In various embodiments, the fusion polypeptide comprises an immunoglobulin type VEGF-A specific therapeutic protein. In various preferred embodiments, the immunoglobulin type VEGF-A specific therapeutic protein is an antibody binding to or with specificity to VEGF-A. In various preferred embodiments, the antibody binding to or with specificity to VEGF-A is a full-length antibody, or a fragment thereof. Such antibody fragments include, but are not limited to, single-chain variable fragments (scFv), single-chain antibodies (scAb), and antigen binding fragments (Fab). The full-length antibody binding to or with specificity to VEGF-A comprises an Fc domain.

In various embodiments, the fusion polypeptide as disclosed herein comprises a non-immunoglobulin type VEGF-A specific therapeutic protein. In various preferred embodiments, the non-immunoglobulin type VEGF-A specific therapeutic protein comprises ligand-binding elements from the extracellular components of VEGFR-1 and/or VEGFR-2. In various preferred embodiments, the ligand-binding elements from the extracellular components of VEGFR-1 and VEGFR-2 comprise the second (Ig) domain of VEGFR-1, and/or the third (Ig) domain of VEGFR-2. Accordingly, in various embodiments of the present invention, a non-immunoglobulin type VEGF-A specific therapeutic protein comprising ligand-binding elements from the extracellular components of VEGFR-1 and/or VEGFR-2 has binding specificity for VEGF-A.

In some embodiments, the therapeutic protein can be a selected from, but not limited to, Aflibercept, Ranibizumab, Bevacizumab, or Brolucizumab, or fragments thereof, or biosimilars.

In some embodiments the VEGF-A specific protein is a therapeutic protein for the treatment of, for example, neovascular eye disease selected from but not limited to the vascular endothelial growth factor-A (VEGF-A) specific recombinant monoclonal antibodies Ranibizumab (tradename Lucentis®) or Bevacizumab (tradename Avastin®) or VEGF-specific single-chain antibody fragment Brolucizumab (RTH258). In some embodiments, the fusion protein comprises Aflibercept as VEGF-A specific protein. In some embodiments, the fusion protein comprises a biosimilar of Aflibercept, for example M710 (Momenta Pharmaceuticals), Mylan NV (Momenta Pharmaceuticals), ALT-L9 (Alteogen), FYB203 (Formycon), CHS-2020 (Coherus BioSciences), or other VEGF antagonists. Other antagonists could be used instead.

In some embodiments the fusion protein comprises a VEGF-A specific protein that is an extracellular domain of a receptor or fragments thereof. In some embodiments VEGF-A specific protein is the recombinant fusion protein Aflibercept (tradename Eylea®) (SEQ ID NO: 15).

In various preferred embodiments, the fusion polypeptide comprises a VEGF-A specific protein, which is a VEGF-A specific fusion protein comprising ligand-binding elements from the extracellular components of VEGFR-1 and/or VEGFR-2. In various embodiments, the VEGF-A specific fusion protein comprises two polypeptide chains, each comprising ligand-binding elements from the extracellular components of VEGFR-1 and/or VEGFR-2. Preferably, the two polypeptide chains are two identical polypeptide chains. In various preferred embodiments, the ligand-binding elements from the extracellular components of VEGFR-1 and VEGFR-2 comprise the second (Ig) domain of VEGFR-1, and/or the third (Ig) domain of VEGFR-2. Accordingly, in various embodiments of the present invention, a fusion protein comprising ligand-binding elements from the extracellular components of VEGFR-1 and/or VEGFR-2 has binding specificity for VEGF-A.

A fusion polypeptide as described above comprising a VEGF-A specific fusion protein comprising ligand-binding elements from the extracellular components of VEGFR-1 and/or VEGFR-2, is capable of inhibiting the activity of VEGF-A. In particular, such a fusion polypeptide prevents binding of VEGF-A to the receptors VEGFR-1 and/or VEGFR-2. More specifically, as disclosed herein, such a fusion polypeptide of the invention prevents binding of VEGF-A to the receptors VEGFR-1 and/or VEGFR-2, and thereby prevents VEGF-A-induced angiogenesis (or prevents activation of the VEGF-A-induced angiogenesis cascade).

In various embodiments, the fusion protein comprises (or is fused to, or linked to) the Fc region (or Fc domain) of an immunoglobulin molecule. Preferably, the Fc region (or Fc domain) is of a human immunoglobulin molecule, more preferably the Fc region (or Fc domain) is of a human IgG1 molecule.

In various embodiments, the fusion polypeptide comprises a VEGF-A specific fusion protein as described above, fused to the Fc domain of an immunoglobulin, wherein each of the two (identical) polypeptide chains of the VEGF-A specific fusion protein has a type II collagen binding protein as described elsewhere herein fused to the Fc domain of each of the two (identical) polypeptide chains. In various embodiments, the type II collagen binding protein fused to each of the two (identical) polypeptide chains of the VEGF-A specific fusion protein is a dimer or multimer, preferably a dimer, of a type II collagen binding protein as described elsewhere herein. The type II collagen binding proteins fused to the two (identical) polypeptide chains of the VEGF-A specific fusion protein may or may not be identical. Preferably, the type II collagen binding proteins fused to the two (identical) polypeptide chains of the VEGF-A specific fusion protein are identical type II collagen binding proteins.

In various preferred embodiments, the VEGF-A specific protein is a fusion protein comprising an amino acid sequence with at least 90%, preferably at least 95%, sequence identity to SEQ ID NO: 15, and wherein the fusion protein exhibits specific binding affinity for VEGF-A, in particular for human VEGF-A. Accordingly, a fusion polypeptide of the invention comprising an amino acid sequence with at least 90%, preferably at least 95%, sequence identity to SEQ ID NO: 15, is capable of inhibiting the the activity of VEGF-A. In particular, as disclosed herein, a fusion polypeptide comprising an amino acid sequence with at least 90%, preferably at least 95%, sequence identity to SEQ ID NO: 15, prevents binding of VEGF-A to the receptors VEGFR-1 and/or VEGFR-2.

Aflibercept, a vascular endothelial growth factor (VEGF) antagonist, is a recombinant dimeric fusion glycoprotein that comprises VEGF binding portions from the extracellular domains of human VEGF receptors 1 and 2, fused to the Fc portion of human IgG₁. Aflibercept prevents VEGF binding to the receptors VEGFR-1 and VEGFR-2, and as a result, suppresses neovascularization and decrease vascular permeability. This ultimately will slow vision loss or the progression of metastatic colorectal cancer. Biosimilars of Aflibercept can be used accordingly.

Various embodiments include a VEGF-A specific protein such as a non-immunoglobulin therapeutic protein. Thus, in some embodiments the fusion protein comprises a) at least 95% identical to SEQ ID NO: 1 that binds to Type II collagen and b) a VEGF-A specific non-immunoglobulin scaffold. Examples may be selected from the group of DARPin (ankyrin repeat protein muteins), Anticalin (lipocalin muteins), Affilin (ubiquitin muteins, proteins with triple-helical structure), Affibody (muteins of the Z-domain of Staphylococcal protein A), Fynomer (mutein of human Fyn SH3 domain), AdNectin (mutein of the tenth domain of human fibronectin), Kunitz domain peptides (muteins of Kunitz domains of various protease inhibitors), Nanofitins (Sac7d muteins), Avimers (muteins of multimerized Low Density Lipoprotein Receptor-A), chagasin scaffold or chagasin-like protease inhibitor proteins, Adnexin scaffold, Centryrin (FN3 domain muteins), Knottin (cysteine-knot miniprotein muteins), Armadillo-repeat protein muteins, Atrimers (tetranectin muteins; C-type lectin domain muteins), CTLA4 based muteins, or others. In some embodiments the fusion protein comprises a therapeutic protein for neovascular eye diseases that is an antagonist, for example, based on a non-immunoglobulin effector moiety. In some embodiments a VEGF-A specific protein for the treatment of, for example, neovascular eye diseases is the non-immunoglobulin binding protein Abicipar (Abicipar pegol). Thus, in some embodiments the fusion protein comprises a) SEQ ID NO: 1 that bind to Type II collagen of the eye and b) Abicipar.

### Structural Characterization of the Fusion Protein or Fusion Polypeptide.

In some embodiments the fusion protein comprises a) Aflibercept (tradename Eylea©) and b) at least 95% identical to SEQ ID NO: 1 that bind to Type II collagen. Thus, in some embodiments for the treatment of neovascular eye diseases the fusion protein comprises a) Ranibizumab and b) at least 95% identical to SEQ ID NO: 1 that bind to Type II collagen. In some embodiments the fusion protein comprises a) Bevacizumab and b) at least 95% identical to SEQ ID NO: 1 that bind to Type II collagen. In some embodiments the fusion protein comprises a) Brolucizumab and b) at least 95% identical to SEQ ID NO: 1 that bind to Type II collagen.

In some embodiments, the fusion polypeptide capable of binding to VEGF-A and Type II collagen that comprises at least two subunits, wherein the first subunit is a binding protein specific for VEGF-A is Aflibercept, and the second subunit is a binding protein of at least 95% identical to SEQ ID NO: 1 specific for Type II collagen. In some embodiments, the fusion polypeptide capable of binding to VEGF-A and Type II collagen that comprises at least two subunits, wherein the first subunit is a binding protein specific for VEGF-A is Aflibercept, and the second subunit is a binding protein of at least 95% identical to SEQ ID NO: 1 specific for Type II collagen wherein the amino acid corresponding to position 25 in SEQ ID NO: 1 is Q, amino acid corresponding to position 29 is Y, and amino acid corresponding to position 33 is W. In some embodiments, the fusion polypeptide capable of binding to VEGF-A and Type II collagen that comprises at least two subunits, wherein the first subunit is a binding protein specific for VEGF-A is Aflibercept, and the second subunit is a binding protein of at least 95% identical to SEQ ID NO: 1 specific for Type II collagen wherein the amino acid corresponding to position 25 in SEQ ID NO: 1 is Q, the amino acid corresponding to position 26 in SEQ ID NO: 1 is Q, the amino acid corresponding to position 27 in SEQ ID NO: 1 is R, the amino acid corresponding to position 28 is N or H, the amino acid corresponding to position 29 is Y, the amino acid corresponding to position 30 is F, the amino acid corresponding to position 31 is R, the amino acid corresponding to position 32 is Q or R, and the amino acid corresponding to position 33 is W.

In some embodiments, the fusion polypeptide capable of binding to VEGF-A and Type II collagen that comprises at least two subunits, wherein the first subunit is a binding protein specific for VEGF-A is Aflibercept, and the second subunit is a binding protein of at least 95% identical to SEQ ID NO: 2 specific for Type II collagen. In some embodiments, the fusion polypeptide capable of binding to VEGF-A and Type II collagen that comprises at least two subunits, wherein the first subunit is a binding protein specific for VEGF-A is Aflibercept, and the second subunit is a binding protein of at least 95% identical to SEQ ID NO: 3 specific for Type II collagen.

In some embodiments the fusion protein includes in N-terminal to C-terminal order the Type II collagen binding protein and VEGF-A specific protein. In other embodiments the fusion protein includes in N-terminal to C-terminal order the VEGF-A specific protein and the Type II collagen binding protein as defined herein. In some embodiments the fusion protein comprises in N- to C-terminal order a) at least 95% identical to SEQ ID NO: 1 that bind to Type II collagen, b) Aflibercept (tradename Eylea©) and c) at least 95% identical to SEQ ID NO: 1 that bind to Type II collagen. Specific, non-limiting examples for Type II collagen binding proteins are at least 95% identical to SEQ ID NO: 2 or SEQ ID NO: 3.

On other embodiments further components can be included N-terminal and/or C-terminal. Further components may be labels or domains for the purpose of purification or to enhance solubility or for stabilization or for detecting, as known to someone skilled in the art.

Functional characterization. One embodiment refers to a fusion protein or fusion polypeptide of the invention comprising a Type II collagen binding protein with binding affinity (KD) of less than 1 μM for Type II collagen. The binding proteins bind Type II collagen with a measurable binding affinity of less than 1 μM, of less than 500 nM, of less than 100 nM, of less than 50 nM, less than 10 nM, less than 5 nM, or less than 1 nM. One embodiment refers to a fusion protein or fusion polypeptide of the invention comprising a VEGF-A binding protein with binding affinity (KD) of less than 1 μM for Type II collagen. In various embodiments, the fusion polypeptide comprising the type II collagen binding protein of the present disclosure has no detectable binding affinity to type I collagen and/or type III collagen. In various other embodiments, the fusion polypeptide comprising the type II collagen binding protein of the present disclosure has no detectable binding affinity to type I collagen, type III collagen, and/or type V collagen. The binding proteins bind VEGF-A (or its isoforms) with a measurable binding affinity of less than 1 μM, of less than 500 nM, of less than 100 nM, of less than 50 nM, less than 10 nM, less than 5 nM, or less than 1 nM.

In some embodiments, the fusion protein binds Type II collagen with binding affinity of less than 1 μM and VEGF-A with binding affinity of less than 50 nM. In other embodiments, the fusion protein binds Type II collagen with binding affinity of less than 10 nM and VEGF-A with binding affinity of less than 10 nM. In some embodiments, the affinities of the fusion protein for Type II collagen and VEGF-A are different. In some embodiments, a low affinity for Type II collagen might be favorable. The appropriate methods are known to those skilled in the art or described in the literature. The methods for determining the binding affinities are known per se and can be selected for instance from the following methods known in the art: enzyme-linked immunosorbent assay (ELISA), surface plasmon resonance (SPR), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Some of the methods are described in the Examples below. Typically, the dissociation constant KD is determined at 20° C., 25° C., or 30° C. The lower the KD value, the greater the binding affinity of the biomolecule for its binding partner. The higher the KD value, the more weakly the binding partners bind to each other.

By specifically binding to Type II collagen in the eye, the collagen specific binding protein may anchor the VEGF-A specific protein to the fibrillar structure of the vitreous humor and thereby ensure an increase of the half-life of the therapeutic protein for eye diseases. In some embodiments the half-life of the VEGF-A binding protein is increased by fusion to a Type II collagen binding protein as disclosed herein. In other embodiments the half-life of the VEGF-A binding protein is increased by at least 50% or even at least 100% by fusion to Type II collagen binding protein as disclosed. In some embodiments the fusion protein as disclosed herein has an increased half-life of at least 1.25-fold, at least 1.5-fold, at least 2-fold, at least 2.5-fold, at least 3-fold, at least 3.5-fold, at least 4-fold or even more relative to the ocular half-life of the therapeutic protein for eye diseases that is not fused to the Type II collagen binding protein as described.

Increases in ocular half-life of a fusion protein compared to the therapeutic protein for eye diseases without fusion to Type II collagen binding protein as described can be determined by administration of the proteins by intravitreal injection and measuring the remaining concentrations at various time points. Concentration can be measured by methods known to some skilled in the art, and include ELISA, mass spectroscopy, western blot, radio-immunoassay, or fluorescent labeling. Methods for pharmocokinetic analysis and determination of half-life and/or mean residence time are known in the art.

In various embodiments, the fusion polypeptide of the invention comprises a VEGF-A specific binding protein and a type II collagen specific binding protein, wherein the VEGF-A specific binding protein exhibits any of the functional properties described elsewhere herein for VEGF-A specific proteins, and wherein the type II collagen specific binding protein exhibits any of the functional properties described elsewhere herein for type II collagen binding proteins. Accordingly, in various embodiments, any of the functional characteristics or properties described herein in relation to a VEGF-A specific binding protein may be combined with any of the functional characteristics or properties described herein in relation to a type II collagen specific binding protein.

Multimers. In some embodiments, the Type II collagen binding protein is a multimer comprising of a plurality of the Type II collagen binding protein as defined herein. A multimer may comprise two, three, four, or more Type II collagen binding proteins. In one embodiment, the Type II collagen binding protein comprises 2, 3, 4, or more Type II collagen binding proteins linked to each other, i.e. the Type II collagen-binding protein can be a dimer, trimer, or tetramer, etc. In some embodiments, the multimer is a dimer of the Type II collagen binding protein as defined above. In some embodiments, the multimeric Type II collagen binding protein may comprise at least two modules of SEQ ID NO: 1. For example, a dimeric Type II collagen binding protein may comprise two monomers of SEQ ID NO: 1 linked to each other in head-to-tail orientation. In some embodiments, the homo-multimeric Type II collagen binding protein may comprise at least two modules of SEQ ID NO: 2. For example, a dimeric Type II collagen binding protein may comprise two monomers of SEQ ID NO: 2 linked to each other in head-to-tail orientation. In some embodiments, the homo-multimeric Type II collagen binding protein may comprise at least two modules of SEQ ID NO: 3. For example, a dimeric Type II collagen binding protein may comprise two monomers of SEQ ID NO: 3 linked to each other in head-to-tail orientation.

In some embodiments the fusion protein comprises a) a type II collagen binding protein at least 95% identical to SEQ ID NO: 1; and b) a VEGF-antagonist, such as Aflibercept or a biosimilar thereof. In some embodiments the fusion protein comprises a) two type II collagen binding protein at least 95% identical to SEQ ID NO: 1; and b) a VEGF-antagonist, such as Aflibercept or a biosimilar thereof. Non-limiting examples are provided in SEQ ID NO: 6 (CID 213182), SEQ ID NO: 7 (CID 213340), SEQ ID NO: 10 (CID 212710), SEQ ID NO: 11 (CID 212708), and SEQ ID NO: 21 (CID 217793). In some embodiments the fusion protein comprises a) three type II collagen binding protein at least 95% identical to SEQ ID NO: 1; and b) a VEGF-antagonist, such as Aflibercept or a biosimilar thereof. In some embodiments the fusion protein comprises a) four type II collagen binding protein at least 95% identical to SEQ ID NO: 1; and b) a VEGF-antagonist, such as Aflibercept or a biosimilar thereof. Non-limiting examples are provided in SEQ ID NO: 8 (CID 213184), SEQ ID NO: 9 (CID 213185), SEQ ID NO: 12 (CID 212709), and SEQ ID NO: 22 (CID 218480). Multimers of the binding protein are generated artificially, generally by recombinant DNA technology well-known to a skilled person.

Linker. In some embodiments the fusion protein or fusion polypeptide as defined above or as defined elsewhere herein can include a linker, for example a polypeptide linker, between the Type II collagen binding protein and the VEGF-A specific protein, or between two or more Type II collagen binding proteins.

The length and composition of a linker may vary between at least one and up to about 50 amino acids. More preferably, the peptide linker has a length of between 1 and 30 amino acids; e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids. It is preferred that the amino acid sequence of the peptide linker is not immunogenic to human beings, stable against proteases and optionally does not form a secondary structure. Suitable amino acids for linkers may be selected but are not limited to from amino acids such as glycine, serine, alanine, or proline.

Use in medicine. Various embodiments relate to the fusion protein as defined above comprising a Type II collagen binding protein and a VEGF-A specific protein for use in the treatment or prevention of neovascular eye diseases. Neovascular eye diseases may be selected from the group of but not limited to neovascular age-related macular degeneration, myopic choroidal neovascularization, idiopathic choroidal neovascularization, choroidal neovascularization, branch retinal vein occlusion, central retinal vein occlusion, diabetic retinopathy, retinopathy of prematurity, diabetic macular edema.

Some embodiments relate to methods for treating a subject with a disorder that affects the eye including administering to the eye a therapeutically effective amount of the fusion protein as defined herein.

The present invention provides a pharmaceutical composition comprising a fusion polypeptide of the invention as described herein, and a therapeutically acceptable carrier and/or diluent.

The present invention provides a fusion polypeptide of the invention as described herein, or a pharmaceutical composition comprising a fusion polypeptide of the invention as described herein, for use in medicine.

The present invention further provides a method for the prevention and/or treatment of VEGF-A-induced (pathologic) angiogenesis in a subject in need thereof, comprising administering to the subject a therapeutically effective amount of a fusion polypeptide of the invention as described herein, or a therapeutically effective amount of a pharmaceutical composition comprising a fusion polypeptide of the invention as described herein. Preferably, the subject is a human subject. In various embodiments, the VEGF-A-induced (pathologic) angiogenesis is VEGF-A-induced (pathologic) ocular angiogenesis. In various embodiments, the method is for the prevention and/or treatment of ocular neovascularization due to pathologic (ocular) angiogenesis, in particular VEGF-A-induced pathologic (ocular) angiogenesis.

The present invention further provides a method for the prevention and/or treatment of an eye disease in a subject in need thereof, comprising administering to the subject a therapeutically effective amount of a fusion polypeptide of the invention as described herein, or a therapeutically effective amount of a pharmaceutical composition comprising a fusion polypeptide of the invention as described herein. Preferably, the subject is a human subject.

In various preferred embodiments, the eye disease is an angiogenic eye disease, more specifically a VEGF-A-associated or VEGF-A-induced angiogenic eye disease. In various other embodiments, the eye disease is a neovascular eye disease, more specifically a VEGF-A-associated or VEGF-A-induced neovascular eye disease. Accordingly, as described herein, the eye disease preferably is a VEGF-A-associated or VEGF-A-induced eye disease. In various embodiments, the eye disease may be any of the eye disease described herein above. In preferred embodiments, the eye disease is any of age-related macular degeneration (AMD), diabetic retinopathy (DR), diabetic macular edema, and retinal vein occlusion.

In various embodiments of the methods for the prevention and/or treatment disclosed herein, the fusion polypeptide of the invention, or the pharmaceutical composition comprising a fusion polypeptide of the invention, is administered by topical administration or by intraocular administration. Preferably, the intraocular administration is via intravitreal route (intravitreal administration).

Compositions. Various embodiments relate to a composition comprising the fusion protein as defined above comprising a Type II collagen binding protein and VEGF-A specific protein. The composition optionally may contain further auxiliary agents and excipients known per se. These include for example but are not limited to stabilizing agents, surface-active agents, salts, buffers, coloring agents etc. The compositions can be in the form of a liquid preparation, a lyophilisates, an aerosol, in the form of powders, granules, in the form of an emulsion or a liposomal preparation.

Various embodiments relate to a pharmaceutical composition for the treatment of diseases affecting the eye comprising a fusion protein as disclosed herein, and a pharmaceutically acceptable carrier and/or diluent. A pharmaceutically acceptable carrier may include solvents, dispersion media, coatings, antibacterial and antifungal agents, isotonic and absorption delaying agents, and the like that are physiologically compatible. The pharmaceutical composition comprising a fusion protein as defined above can be used for treatment or diagnosis of neovascular eye diseases. The pharmaceutical composition may be suitable for intravitreal administration.

The composition for the treatment of eye disorders should be fluid and sterile. In some cases, isotonic agents, polyalcohols, and sodium chloride may be included in the composition. The composition may include an agent which delays absorption, for example, gelatin or aluminum monostearate. The type of pharmaceutical preparation depends on the type of eye disease to be treated, the route of administration, the severity of the disease, the patient to be treated and other factors known to those skilled in the art of medicine.

The compositions contain a therapeutically or diagnostically effective dose of the fusion protein as defined above. The amount of fusion protein to be administered depends on the organism to be treated, the type of disease, the age and weight of the patient and further factors known per se. Depending on the galenic preparation these compositions can be administered by injection, or by other conventionally employed methods of application for the treatment of eye diseases. In various embodiments, the composition is suitable for delivery to the eye by intravitreal, topical ophthalmic, intraretinal, subretinal, suprachoroidal and intracameral delivery.

Various embodiments relate to a method for treating or diagnosing a subject with an eye disease, comprising administering to the eye of the subject a fusion protein as defined above. Preferred is a method for treating a subject with an eye disease selected from neovascular eye diseases and inflammatory eye diseases.

Preparation of fusion proteins. A fusion protein as defined above may be prepared by any of the many conventional and well-known techniques such as plain organic synthetic strategies, solid phase-assisted synthesis techniques, fragment ligation techniques or by commercially available automated synthesizers. On the other hand, they may also be prepared by conventional recombinant techniques alone or in combination with conventional synthetic techniques. Furthermore, they may also be prepared by cell-free in vitro transcription/translation or in combination with conventional synthetic techniques.

Various embodiments relate to an isolated polynucleotide encoding a fusion protein as defined above. The invention also encompasses polypeptides encoded by said polynucleotides. The invention further provides an expression vector comprising said polynucleotide, and a host cell comprising said polynucleotide or said expression vector.

Various embodiments relate to a method for the production of a fusion protein as defined above comprising culturing of a host cell under suitable conditions in order to obtain said fusion protein and optionally isolating said fusion protein.

Various embodiments relate to a polynucleotide encoding a fusion protein as described above. The invention further provides an expression vector comprising said polynucleotide, and a host cell comprising said isolated polynucleotide or the expression vector.

Various embodiments relate to a method for the production of a fusion protein as described above comprising culturing of a host cell under suitable conditions which allow expression of said fusion protein and optionally isolating said fusion protein.

For example, one or more polynucleotides which encode for fusion protein may be expressed in a suitable host and the produced fusion protein can be isolated. Vectors comprising said polynucleotides are covered herein. A further embodiment relates to a vector comprising said nucleic acid molecule. A vector means any molecule or entity (e.g., nucleic acid, plasmid, bacteriophage or virus) that can be used to transfer protein coding information into a host cell. Furthermore, an isolated cell is disclosed comprising said nucleic acid molecule or said vector. Suitable host cells include prokaryotes or eukaryotes. Various mammalian or insect cell culture systems can also be employed to express recombinant proteins.

An embodiment also relates to a host cell or a non-human host carrying said vector. A host cell is a cell that has been transformed with a nucleic acid sequence and thereby expresses a gene of interest.

Suitable conditions for culturing prokaryotic or eukaryotic host cells are well known to the person skilled in the art. Cultivation of cells and protein expression for the purpose of protein production can be performed at any scale, starting from small volume shaker flasks to large fermenters, applying technologies well-known to any skilled in the art.

One embodiment is directed to a method for the preparation of a binding protein as detailed above, said method comprising the following steps: (a) preparing a nucleic acid encoding a fusion protein as described above; (b) introducing said nucleic acid into an expression vector; (c) introducing said expression vector into a host cell; (d) cultivating the host cell; (e) subjecting the host cell to culturing conditions under which fusion protein is expressed, thereby producing a fusion protein as defined herein; (f) optionally isolating the fusion binding protein produced in step (e); and (g) optionally conjugating the fusion protein with further functional moieties as defined herein.

In general, isolation of purified fusion protein from the cultivation mixture can be performed applying conventional methods and technologies well known in the art, such as centrifugation, precipitation, flocculation, different embodiments of chromatography, filtration, dialysis, concentration and combinations thereof, and others. Chromatographic methods are well-known in the art and comprise without limitation ion exchange chromatography, gel filtration chromatography (size exclusion chromatography), hydrophobic interaction chromatography, or affinity chromatography.

For simplified purification, the fusion protein can be fused to other peptide sequences having an increased affinity to separation materials. Preferably, such fusions are selected that do not have a detrimental effect on the functionality of the fusion protein or can be separated after the purification due to the introduction of specific protease cleavage sites. Such methods are also known to those skilled in the art.

### EXAMPLES

The following Examples are provided for further illustration of the invention. The invention, however, is not limited thereto, and the following Examples merely show the practicability of the invention on the basis of the above description. For a complete disclosure of the invention reference is made also to the literature cited in the application which is incorporated completely into the application by reference.

### Example 1. Mammalian Expression of Fusion Proteins

Expi293-F-cells were cultured with 0.5-1 Mio cells/ml in Expi293-F Expression medium (Fisher scientific, 13489756) in shake flasks with 135 rpm, at 37° C., 8% CO2 and 95% humidity. One day before transfection, cells were seeded with a density of 2.0 Mio/cells/ml. On the day of transfection, cells were seeded with a density of 2.5 Mio/ml. 1 μg plasmid-DNA of CID 212708, CID 212709, CID 212710, CID 213182, CID 213184, CID 213340, CID 217793, CID 218480, and control fusion protein not binding Type II collagen 2 (CID 213350) per ml of culture volume were used and diluted in Opti-MEM® I Reduced Serum Medium (Life Technologies, 31985-062). ExpiFectamine™ was diluted in Opti-MEM® I Reduced Serum Medium, according to manufacturer information and incubated for 5 min at rt. Subsequently, DNA-solution was added to the ExpiFectamine-mixture and incubated for 20 min. at rt, before adding to the cells. After 16 h, Enhancer was added to the cells. Supernatant was collected after 96-120 h, centrifuged and filtered through 0.45 μm membrane.

### Example 2. Purification of Fusion Proteins

Fusion proteins were purified via MabSelect SuRe (Cytiva) affinity chromatography followed by a size exclusion chromatography (SEC) has been performed using an Åkta system and a Superdex™ 200 HiLoad 26/600 column (Cytiva). All chromatography processing steps were carried out on AKTA systems using matrices from Cytiva. Homogenous species of all purified ligands were obtained; no aggregation was detected. Following SDS-PAGE analysis positive fractions were pooled and their protein concentrations were measured. Finally including endotoxins were reduced by using Pierce High Capacity Endotoxin Removal Spin Columns (ThermoFisher). Further analysis included SDS-PAGE, LAL test, SE-HPLC and RP-HPLC. Protein concentrations were determined by absorbance measurement at 280 nm using the molar absorbent coefficient. Reversed phase chromatography (RP-HPLC) has been performed using a Thermo HPLC system and a PLRP-S(5 μm, 300 Å) column (Agilent). Analytic size exclusion chromatography (SE-HPLC) has been performed using a Thermo HPLC system and a Superdex200 increase 5/150 GL (Cytiva). The evaluation of the endotoxin level was analyzed using LAL test cartridges (Charles River). All fusion proteins were successfully purified with sufficient endotoxin values (see Table 1 for results).

### Example 3. Binding Analysis of Fusion Proteins by SPR

Binding of Type II collagen or VEGF-A was evaluated by SPR measurements using Biacore 3000 (GE Healthcare). Purified Type II collagen was obtained by Chondrex.inc and VEGF-A (VEGF-165) was obtained by Millipore. The purified target proteins were immobilized on a CM-5 sensor chip (GE Healthcare) using NHS/EDC after PDEA activation resulting in about 1000 RU. The chip was equilibrated with SPR running buffer (PBS 0.05% Tween pH 7.3).

The analytes were applied to the chip in serial dilutions with a flow rate of 30 μl/min. The association was performed for 120 seconds and the dissociation for 120 seconds. After each run, the chip surface was regenerated with 30 μl regeneration buffer (10 mM glycine pH 2.0) and equilibrated with running buffer. Upon binding, fusion proteins were accumulated on the surface increasing the refractive index. This change in the refractive index was measured in real time and plotted as response or resonance units versus time. Data evaluation was operated via the BIAevaluation 3.0 software, provided by the manufacturer, by the use of the Langmuir 1:1 model (RI=0). Evaluated dissociation constants (K_{D}) were standardized against the immobilized fusion protein and indicated. Shown is the change in refractive index measured in real time and plotted as response or resonance unit [RU] versus time [sec]. Results are shown in Table 1.

TABLE 1A
Analytics of fusion proteins (purity and affinity) (N-terminal: Aflibercept; C-
terminal: Col2-binding protein)

| Fusion protein | | | | | | | | | |
|---|---|---|---|---|---|---|---|---|---|
| | SEQ | | Purity | | | Affinity (SPR) vs | | | |
| | ID | | SE-HPLC | RP-HPLC | Endotoxin | hCol2 | pCol2 | rbCol2 | VEGF-A |
| CID | NO: | CONSTRUCT | (%) | (%) | (EU/ml) | (nM) | (nM) | (nM) | (nM) |
| 213182 | 6 | n[VEGF-BP, Col2- | 100 | 100 | 0.25 | 1.38 | 1.15 | 1.05 | 2.2 |
| | | BP1as dimer]c | | | | | | | |
| 213184 | 8 | n[VEGF-BP, Col2-BP1 | 100 | 100 | 0.77 | 0.15 | 0.39 | 0.52 | 4.14 |
| | | as tetramer]c | | | | | | | |
| 212710 | 10 | n[VEGF-BP, Col2-BP2 | 100 | 100 | 0.47 | 4.71 | 6.25 | 4.0 | 2.54 |
| | | as dimer]c | | | | | | | |
| 213350 | 13 | n[VEGF-BP, non Col2 | 100 | 100 | <0.2 | no binding | no binding | no binding | 1.93 |
| | | BP PAdelFc as dimer]c | | | | | | | |

VEGF-BP refers to the VEGF-A binding protein Aflibercept. Col2-BP refers to Type II collagen binding protein of SEQ ID NO: 1, in particular Col2-BP1 refers to the Type II collagen binding protein of SEQ ID NO: 2; Col2-BP1 refers to the Type II collagen binding protein of SEQ ID NO: 3. The control fusion protein is a construct of Aflibercept fused to a dimer of a non-Type 11 collagen-binding protein (non-Col2 BP) of SEQ ID NO: 14. hCol2 refers to human Type 11 collagen, pCol2 refers to pig Type 11 collagen, and rbCol2 refers to rabbit Type 11 collagen.

TABLE 1B
Analytics of fusion proteins (purity and affinity)((N-terminal
part: Col2-binding protein; C-terminal part: N- and C-terminal
Col2-Binding protein, Aflibercept in the middle)

| | SEQ | | Purity | | Affinity (SPR) vs | |
|---|---|---|---|---|---|---|
| | ID | | SE-HPLC | RP-HPLC | hCol2 | VEGF-A |
| CID | NO: | Fusion protein | (%) | (%) | (nM) | (nM) |
| 217793 | 21 | n[Col2-BP1_as dimer - | 100 | 96 | 35 | 2 |
| | | VEGF-BP]c | | | | |
| 218480 | 22 | n[Col2-BP1_as dimer, | 100 | 100 | <50 | 9 |
| | | VEGF-BP - Col2-BP1 | | | | |
| | | as dimer]c | | | | |

### Example 4. KD-Determination of Binding Proteins (ELISA)

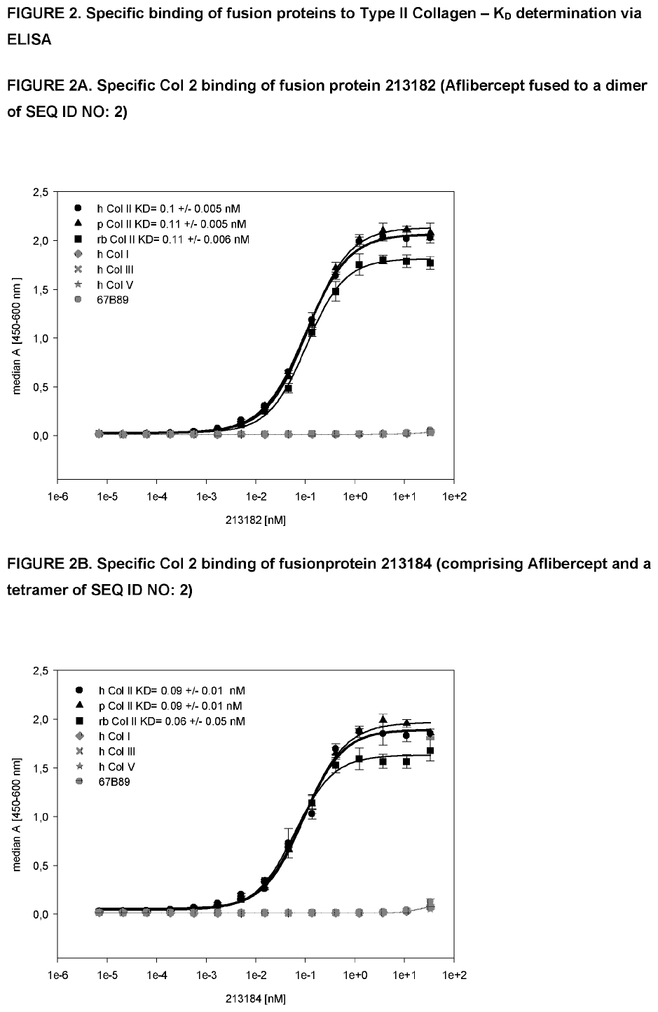

High binding plates (Greiner, 781061) were immobilized with 2.5 μg/ml recombinant human Type II collagen (hColll, Chondrex, CHX-20051), rabbit Type II collagen (rbColll, prepared from rabbit eyes), porcine Type 11 collagen (pColll, prepared from porcine eyes), human Type I collagen (hColl, Sigma, C5483-1MG), human Type V collagen (hCoIV, Abcam, ab7537), human Type III collagen (hCollll, Abcam, ab7535) and 67B89 (domain of fibronectin, produced in E. coli) over night at 4° C. Fusion proteins CID 212709, CID 213182, CID 213184 and CID 213340 were analyzed with a dilution series in a range of 1111 nM to 6.9×10⁻⁵ nM. ELISA-plates were washed 3 times with PBST (PBS+0.1% Tween) and blocked with 3% BSA/0.5% Tween/PBS 2 h at RT. Plates were washed with PBST and incubated with a-human-IgG-antibody at a dilution of 1:10000. Results. CID 212709 (SEQ ID NO: 12), CID 213182 (SEQ ID NO: 6), CID 213184 (SEQ ID NO: 8) and CID 213340 (SEQ ID NO: 7) show a strong specific binding to Type II collagen with a K_{D} of 2-3-digit pM. No unspecific binding to Type I collagen, Type III collagen, Type V collagen or to a domain of fibronectin (67B89) was detected (FIG. 2; the result for CID 213340 is comparable to CID 213182 but not shown in the figure).

### Example 5: Analysis of Specific Binding of Proteins by IHC

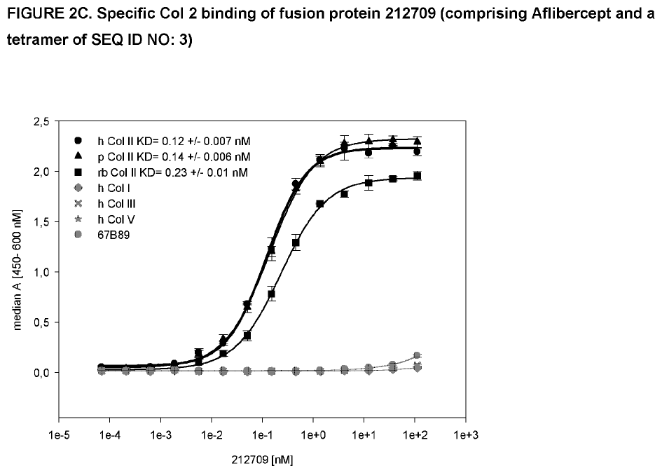

Sections of paraffin-embedded rabbit and pig eyes were deparaffinated with xylene and rehydrated with a descending concentration series of ethanol. For antigen retrieval slices were incubated in TE-buffer, pH 9.0 for 5 min. at 110° C. After blocking of endogenous peroxidases with peroxidase blocking solution (Novolink Polymer™ Detection System, Leica, RE7140-K) slices were incubated with 5 μM, 2.5 μM or 500 nM of CID 213184 for 45 min at rt. CID 213184 is a fusion protein comprising SEQ ID NO: 2 as tetramer and Aflibercept; Aflibercept is located N-terminal, the tetramer of SEQ ID NO: 2 is C-terminal. CID 213184 was analyzed with goat-anti-human-IgG-POD (Abcam, ab98624) with a dilution of 1:200. Positive control was a goat-anti-Type II collagen antibody (Biomol, #ARG20787) at 2 pg/ml and a donkey-anti-goat-IgGFc (HRP)-antibody (Abcam, #ab97110) at dilution of 1:200. Tissue slices were incubated with antibodies for 30 min. Washing steps were done with TBS pH 7.6. For visualization, AEC-staining (Dako, K3461) was done and after washing with water, nuclei were stained with Mayers Hamalum solution (Merck, 1.09249.0500). Results are shown in FIG. 3. Similar results were obtained with fusion protein CID 213182.

### Example 6: Mammalian Expression of Fusion Proteins for Animal Study

Cells were cultured in ExpiCHO™ Expression Medium (Thermo Fisher Scientific, A2910001) with 0.5 Mio cells/ml. Cells were seeded with a density of 4.0 Mio/ml, one day before transfection. For transfection, cells were seeded with 6.0 Mio/ml. 1 pg plasmid-DNA of CID 215631, CID 213182, CID 213184, CID 212710 and of the control fusion protein (CID 213350) per ml of culture volume were diluted in OptiPRO™ SFM (Thermo Fisher Scientific, 12309019). ExpiFectamine™ (Thermo Fisher, 29129) were diluted in OptiPRO™ SFM and mixed with DNA solution. After 3 min of incubation at rt, the transfection mixture was added to the cells. After incubation for 24 h at 37° C. ExpiFectamine™ Enhancer and ExpiFectamine™ Feed were added and cells incubated at 32° C. Supernatant was collected after 120-168 h, centrifuged and filtered through 0.45 μm membrane.

### Example 7: Efficacy in the Rabbit VEGF Leakage Model

The objective of this study was to determine the efficacy and tolerability of fusion proteins CID 213182 (SEQ ID NO: 6) and CID 212710 (SEQ ID NO: 10), compared to Aflibercept without fusion to a Col2-binding protein in a rabbit VEGF leakage model. The fusion proteins and the controls (Aflibercept and vehicle) were administered on Day −1, and retinal leakage was induced with VEGF injected intravitreal in the right eye on Day 0. Ocular examinations and examination of intraocular pressure were performed at baseline and on Days 3, 7, and 14. The retina was imaged at baseline and on Days 3, 7, and 14, and fluorescein angiography was performed at baseline and on Day 3 to assess retinal leakage.

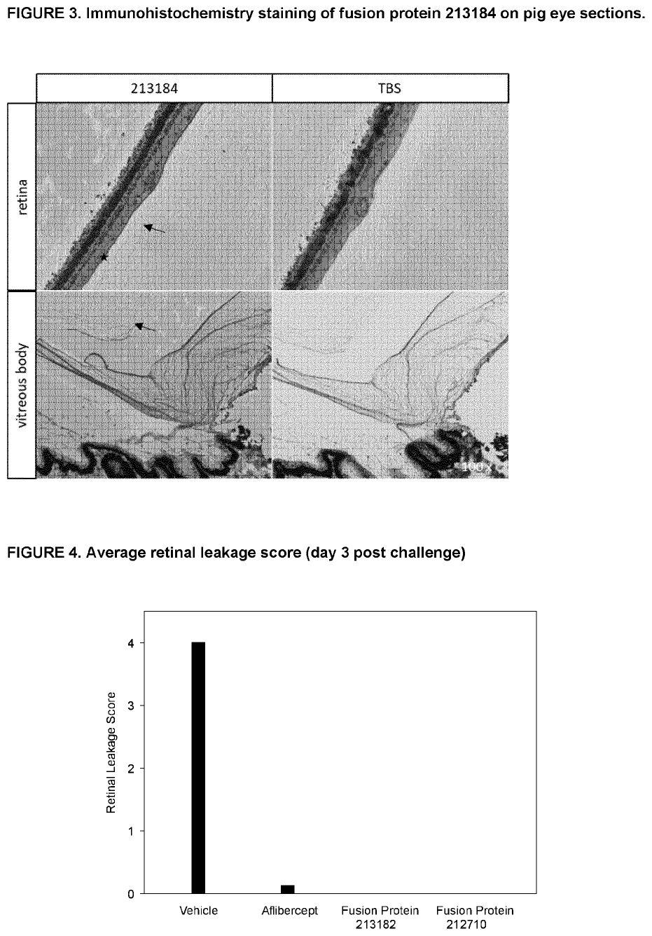

The animals in all groups gained a normal amount of body weight over the course of the study. The results show that all materials were well tolerated. Retinal leakage was assessed with fluorescein angiography. The vehicle (without Aflibercept or fusion protein) demonstrates severe retinal vascular leakage at Day 3, however, all other treatments indicate strong efficacy in this model (FIG. 4).

The results suggest that all fusion proteins and controls are well-tolerated in an animal model (male Dutch Belted rabbits (Oryctolagus cuniculus)), and treatment with the fusion proteins prevents retinal vascular leakage induced by VEGF administration compared to vehicle treatment. The results may also suggest that fusion proteins of the invention exhibit an increased efficacy as compared to Aflibercept without fusion to a Type II collagen binding protein.

1. A fusion polypeptide capable of binding to VEGF-A and Type II collagen that comprises at least two subunits, wherein the first subunit is a binding protein specific for VEGF-A, and the second subunit is a binding protein specific for Type II Collagen, and wherein the second subunit comprises at least one amino acid sequence with at least 95% sequence identity to SEQ ID NO: 1.

2. The fusion polypeptide of claim 1, wherein the first subunit comprises a full-length immunoglobulin or an antigen-binding domain thereof or an extracellular domain of a receptor or fragments thereof having binding affinity for VEGF-A.

3. The fusion polypeptide of claim 1, wherein the first subunit is selected from Aflibercept, Ranibizumab, Bevacizumab, or Brolucizumab, or fragments thereof, or biosimilars thereof.

4. The fusion polypeptide of claim 1, wherein the second subunit comprises at least one amino acid sequence with at least 95% sequence identity to SEQ ID NO: 2, or wherein the second subunit comprises at least one amino acid sequence with at least 95% sequence identity to SEQ ID NO: 3.

5. The fusion polypeptide of claim 1, wherein the second subunit comprises a multimer of an amino acid sequence with at least 95% sequence identity to SEQ ID NO: 1, SEQ ID NO: 2, or SEQ ID NO: 3.

6. The fusion polypeptide of claim 1, wherein the fusion polypeptide does not bind to Type I collagen, Type III collagen, or Type V collagen.

7. The fusion polypeptide of claim 1, wherein the first subunit is linked to the second subunit via a linker.

8. The fusion polypeptide of claim 7, wherein the fusion polypeptide comprises at least two subunits in N-terminal to C-terminal orientation, wherein the subunit at the N-terminus is specific for VEGF-A, and the subunit at the C-terminus is specific for Type II Collagen.

9. The fusion polypeptide of claim 1, wherein the subunit at the N-terminus is specific for Type II Collagen, and the subunit at the C-terminus is specific for VEGF-A.

10. The fusion polypeptide of claim 1, wherein the fusion polypeptide specifically binds to human VEGF-A and human Type II Collagen.

11. (canceled)

12. The fusion protein of claim 1, wherein the Type II Collagen binding protein increases the half-life of the binding protein specific for VEGF-A at least 1.5 fold as compared to the binding protein specific for VEGF-A without fusion to the binding protein for Type II Collagen.

13. A pharmaceutical composition for the treatment of eye diseases comprising the fusion polypeptide of claim 1 and a therapeutically acceptable carrier and/or diluent.

14. A method for producing the fusion polypeptide of claim 1 comprising culturing of a host cell that expresses the fusion polypeptide of claim 1 under suitable conditions in order to obtain said fusion protein and optionally isolating said fusion polypeptide.

15. A method for treating a neovascular eye disease, the method comprising administering to a subject in need thereof the fusion protein of claim 1 in an amount and via a route sufficient to treat the neovascular eye disease.

16. A host cell comprising a polynucleotide that encodes the fusion protein of claim 1 or an expression construct that directs expression of the fusion protein of claim 1 in the host cell.
